(19) United States
(12) Patent Application Publication
(10) Pub. No.: US 2026/0240582 A1
(43) Pub. Date: Aug. 20, 2026

(54) Kyphoplasty System

(71) Applicant: Lentek, LLC, Palm Beach Gardens, FL (US)

(72) Inventor: Sergio Lenchig, Palm Beach Gardens, FL (US)

(73) Assignee: Lentek, LLC, Palm Beach Gardens, FL (US)

(21) Appl. No.: 19/330,692

(22) Filed: Sep. 16, 2025

Related U.S. Application Data
(60) Provisional application No. 63/759,625, filed on Feb. 18, 2025.

(51) Int. Cl. A61B 17/88

(52) U.S. Cl. CPC A61B 17/8855

(57) ABSTRACT

The devices and methods described below provide for kyphoplasty procedure that is quicker and safer than current kyphoplasty methods. The method entails insertion of an asymmetric balloon catheter, with a balloon expansion limited to a minor arc relative to the catheter tube (rather than entirely encircling the catheter tube), into a crushed vertebral body with the balloon primary plane of expansion (the width of the balloon) aligned with a first plane crossing the vertebral body, thereafter inflating the balloon to deform cancellous bone within the vertebral body and create a void, followed by deflating the balloon, reorienting the balloon with the balloon primary plane of expansion (the width of the balloon) aligned with a second plane crossing the vertebral body to create a second void or enlarge the first void.

This application claims priority to U.S. Provisional Application 63/759,625, filed Feb. 18, 2025, which is pending.

### FIELD OF THE INVENTIONS

The inventions described below relate to the field of kyphoplasty.

### BACKGROUND OF THE INVENTIONS

Kyphoplasty is a procedure used to treat compression fractures of the vertebrae, to stabilize the compression fracture, restore the height of the vertebrae, and prevent collapse. In the procedure, a surgeon inserts a balloon into the vertebral body, through the cortical shell into the cancellous bone within the cortical shell. The surgeon then inflates the balloon to displace cancellous bone, jack up the vertical body and restore the height of the vertical body, and then removes the balloon and injects cement into the cavity created by the balloon.

In kyphoplasty procedures, achieving optimal vertebral height restoration often requires precise placement and controlled expansion of the balloon within the fractured vertebral body. However, in many cases, the initial positioning of the balloon may not result in the desired kyphotic correction, necessitating repositioning of the balloon to achieve better vertebral height restoration and load distribution. Existing kyphoplasty balloon systems, however, offer limited or no capability for in-situ repositioning once the balloon has been deployed within the vertebral body. As a result, surgeons are often forced to withdraw the balloon entirely, reposition the trocar, and reintroduce the balloon, which introduces several procedural and safety challenges. One major drawback of this removal and reinsertion process is the significant stress it places on the balloon material itself. Additionally, repositioning the balloon through trocar removal and reentry often requires making a second incision to introduce the trocar into a new location within the pedicle, which introduces more surface area for potential bacterial contamination. Moreover, in many cases, the balloon cannot simply be withdrawn and reinserted without concerns about contamination. Once a balloon has been deployed and exposed to bodily fluids, reinserting it increases the risk of introducing contaminants into the vertebral body, potentially leading to postprocedural infections. Thus, current kyphoplasty balloon systems lack an effective means of repositioning the balloon within the vertebral body without requiring full withdrawal and reentry, leading to increased procedural complexity, lengthier operating times, lengthier exposure to fluoroscopy, higher risk of complications, and additional costs.

### SUMMARY

The devices and methods described below provide for kyphoplasty procedure that is quicker and safer than current kyphoplasty methods. The method entails insertion of an asymmetric balloon catheter, with a balloon expansion limited to a minor arc relative to the catheter tube (rather than entirely encircling the catheter tube), into a crushed vertebral body with the balloon primary plane of expansion (the width of the balloon) aligned with a first plane crossing the vertebral body, thereafter inflating the balloon to deform cancellous bone within the vertebral body and create a void, followed by deflating the balloon, reorienting the balloon with the balloon primary plane of expansion (the width of the balloon) aligned with a second plane crossing the vertebral body to create a second void or enlarge the first void.

### BRIEF DESCRIPTION OF THE DRAWINGS

FIG. 1 illustrates the kyphoplasty balloon catheter system comprising a catheter tube and a balloon disposed on the distal end of the catheter tube.

FIGS. 2, 3 and 4 illustrate the geometry of the balloon that may be used in the system of FIG. 1.

FIGS. 5 and 6 illustrate placement of the balloon within the vertebral body.

FIG. 7 is a side view/vertical cross-section of a collapsed vertebral body.

FIG. 8 illustrates inflation of the balloon within the vertebral body after a first inflation of the balloon

FIG. 9 is a side view/vertical cross-section of the vertebral body after a second inflation of the balloon.

FIG. 10 is an illustration of the backing bar of FIG. 2.

FIG. 11 is an illustration of a locking device for use with the balloon catheter.

FIG. 12 illustrates an arrangement of a radiopaque cover for the balloon.

### DETAILED DESCRIPTION OF THE INVENTIONS

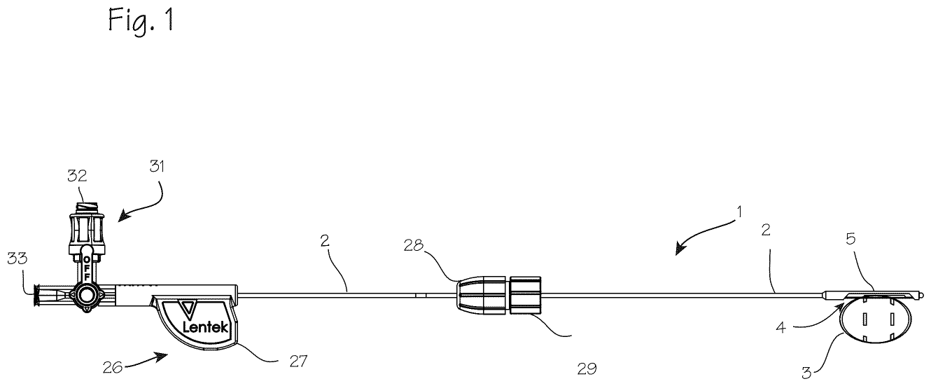

FIG. 1 illustrates the kyphoplasty balloon catheter 1 comprising a catheter tube 2 and a balloon 3 disposed within the distal end 2d of the catheter tube. The catheter tube includes a side-facing aperture 4 which allows the balloon to expand away from the catheter in a single direction, while an intact portion 5 of the tube inhibits expansion of the balloon in a direction opposite the side aperture. A lumen of the catheter tube provides fluid communication from the catheter tube proximal end 2p to the balloon. The balloon is expandable, such that it extends radially through the side aperture.

The balloon is configured to create an intraosseous cavity to one side of the distal end of the catheter tube and centered on a plane. In use the balloon will be oriented to expand medially from the tube, to transect the vertebral body, along a plane which may be a transverse plane/cross section of the vertebral body, or a coronal plane/cross section of the vertebral body, or a slanted cross-section.

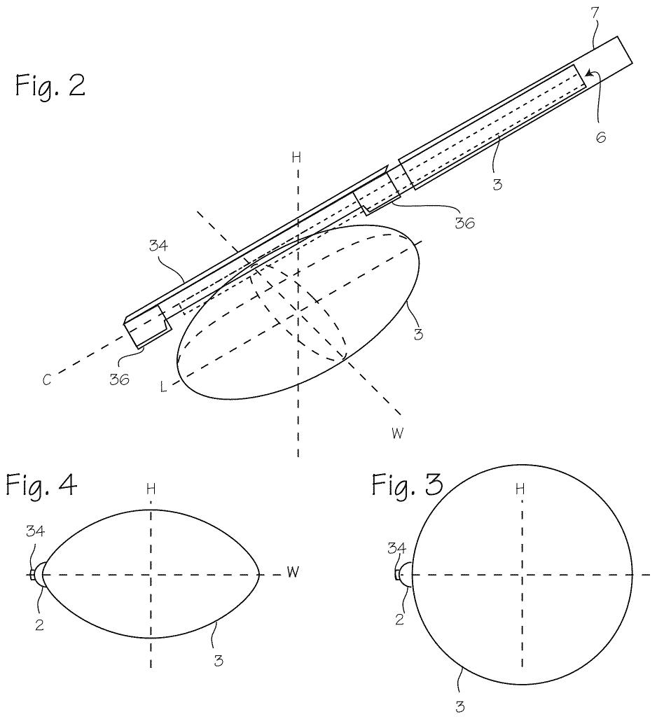

As shown in FIG. 2, the balloon, disposed on the distal end of the catheter, is characterized by a long axis L, a width axis W measured from the catheter and perpendicular to the long central axis of the catheter, and a height axis H displaced from the long central axis of the catheter and perpendicular to the long central axis of the catheter and the long axis of the balloon, wherein the long axis of the balloon, when inflated, is displaced from the long axis of the catheter. When un-inflated or stowed for delivery within a cannula, the long axis of the balloon is roughly coaxial with the long axis of the catheter tube. The height and width axis may loosely correspond to the superior/inferior axis of the vertebral body and the medial/lateral axis of the vertebral body. The balloon may be an ellipsoid, an oblate spheroid (pancake) or a prolate spheroid. The balloon preferably comprises an ellipsoid with a long axis, a major axis (width) and a minor axis (height) in which the major axis is longer than the minor axis (as in a plane-strain ellipsoid). The balloon may comprise an ellipsoid with a polar axis/long axis parallel to the catheter tube, a long width axis and a short height axis (as in an oblate ellipsoid). The balloon may comprise a prolate spheroid/ellipsoid (rugby ball), with a polar axis (the long axis), a minor axis (height axis) and major axis (width axis) both of the same length shorter than the polar axis. The balloon may comprise an entire ellipsoid or a partial ellipsoid. For each shape, the balloon, when expanded, is disposed on one side of the catheter tube. As shown in FIG. 2, the balloon is disposed on the distal end 2d of the catheter 2, with the long axis L parallel to the long central axis C of the catheter, a width axis W measured from the catheter and perpendicular to the long central axis C of the catheter, and a height axis H displaced from the long central axis of the catheter and perpendicular to the long central axis of the catheter and the long axis of the balloon. The long axis of the balloon, when inflated, is displaced from the long axis of the catheter (this establishes the lopsided balloon, without ambiguity). The balloon is in fluid communication with the lumen 6 of the catheter, which in turn is in fluid communication with an insufflator operable (through 3-way valve 31) to push fluid into the balloon to inflate the balloon.

The balloon catheter is configured to limit expansion of the balloon to a minor arc about the catheter tube, whereby the balloon expands away from the catheter tube, limited to a minor arc of the circumference of the catheter tube, rather than uniformly about the catheter tube.

Also shown in FIG. 2 is a trocar 7, used to gain access to the inside of the vertebral body. The balloon catheter can be advanced through the trocar, and rotated within the trocar, to place the balloon within the cancellous bone.

FIGS. 3 and 4 are radial cross-sections of the balloon, at a longitudinal center of the balloon. FIG. 3 depicts a generally circular cross section, with a height equal to the width, such that the balloon, when inflated, is shaped like a rugby ball (the prolate spheroid/ellipsoid mentioned above), while FIG. 4 depicts a ovoid cross section, such that the balloon is shaped like a flattened rugby ball, with a width that is substantially longer than the height. The configuration of FIG. 4 may be a final configuration achieved at full inflation pressure, or an intermediate configuration of the balloon shown in FIG. 3, achieved through partial inflation. The degree of inflation will likely vary as needed to create voids as desired by the surgeon.

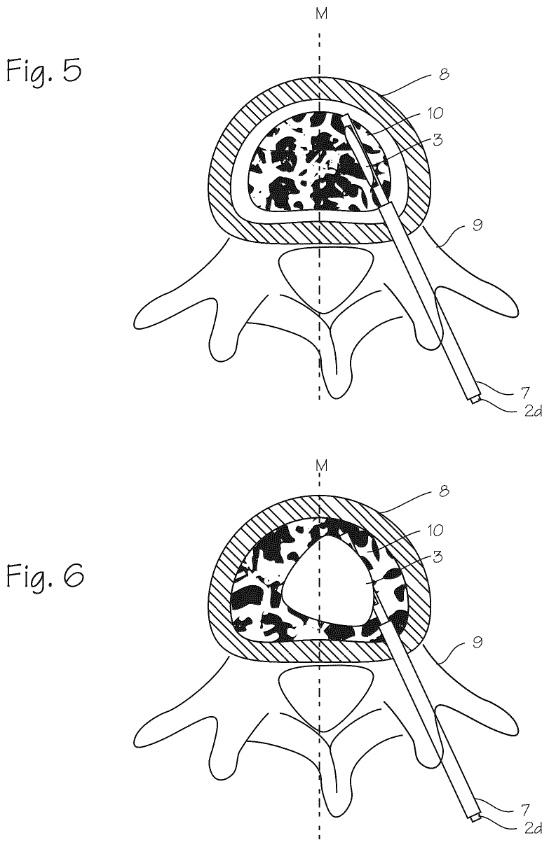

FIG. 5 illustrates placement of the balloon 3 within the vertebral body. A surgeon has hammered the trocar 7 through the cortical shell 8, preferably through the pedicle 9 on one side of the vertebral body, and the distal end 2d of the balloon catheter has been advanced through the trocar so that the distal end of the catheter tube, and the balloon, is inside the cortical shell of the vertebral body and amidst the cancellous bone 10 of the vertebral body such that the long axis of the catheter tube is proximate a lateral area of the cortical body.

The catheter distal end is also oriented such that the side aperture is directed toward a medial area of the vertebral body, and the balloon is expandable toward a medial area of the vertebral body, with the width axis disposed on a first radial line relative to the catheter tube from the catheter tube central axis toward the medial area of the vertebral body. The radial line lies in a first plane transecting the vertebral body.

FIG. 6 illustrates inflation of the balloon with the vertebral body. Using an insufflator secured to the inflation port of the three-way valve, the surgeon has forced fluid into the balloon to inflate the balloon. The balloon is sized and shaped, relative to the vertebral body, such that it may be inflated to extend from the catheter, in a medial direction from the catheter, such that the balloon extends across the vertical midline M. The size of the balloon will vary depending on the size of the vertebra to be treated. The balloon may be manufactured in varying sizes, depending on the size of the vertebra to be treated. Once installed in the vertebral body, the balloon can be inflated to varying sizes depending on how big the surgeon wants to make the void. For example, a balloon can be manufactured at 20 mm long, 17 mm wide such that when inflated with 4cc it expands toward an unconstrained size of 17 mm width×20 mm long, and when inflated with 5 cc the balloon expands toward an unconstrained size of 18.5 mm width×20 mm length, to provide a balloon appropriately sized for the desired expanded size in typical vertebrae.

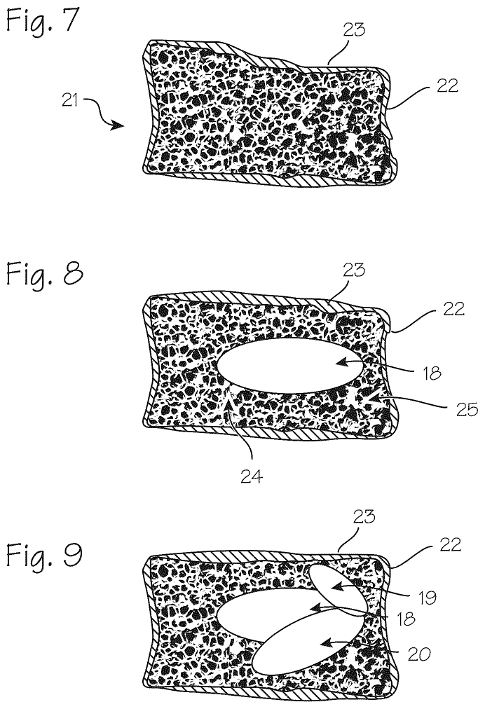

FIG. 7 is a side/lateral view/vertical cross-section of a collapsed vertebral body 21, which may be the result of aging, disease or injury. This illustration shows a fractured side wall 22 and a superior endplate 23 that has collapsed, so that the vertebral body is shorter than it should be, and lopsided.

FIG. 8 illustrates inflation of the balloon within the vertebral body. With the trocar inserted through the pedicle and the balloon catheter distal end with the balloon inside the cortical body, and positioned within the cancellous bone, the surgeon has secured an insufflator to the proximal end of the cannula tube, and pumped a fluid into the balloon, through the catheter tube, to inflate the balloon to displace cancellous bone, and create a cavity 18 within the cancellous bone (and, preferably, at least partially restore the height of the vertebral body). Inflating the balloon within the vertebral body, inside the trabecular/cancellous bone, such that the balloon expands away from the long axis of the catheter tube and into an area of the cancellous bone medial to the catheter tube (marked as item 24) but does not expand into the cancellous bone lateral to the catheter tube (marked as item 25), creates an intraosseous cavity medial to the distal end of the catheter tube and centered on a first plane or cross section of the vertebral body. The cavity created is illustrated in FIG. 8, which is a side view/vertical cross-section of the vertebral body. Also, due to the lifting effect of the balloon, inflation of the balloon has resulted in lifting the endplate towards its original height.

Surgeons will generally prefer a larger void, or more voids, in the cancellous bone than can be created in a single inflation. Surgeons may also prefer to lift the superior endplate further than can be accomplished with a single inflation. To achieve this under currently known procedures, surgeons repeat the process on the other side of the vertebral body, which requires another incision and penetration of the body, including hammering another trocar into the vertebral body, and inserting another balloon. This additional trauma can be avoided with the balloon catheter system of the earlier figures by (1) withdrawing fluid from the balloon after creating the first void, (2) leaving the balloon in place within the cancellous bone, and (3) rotating the catheter tube to point the side aperture (and the width axis of the balloon) in a second direction (different from the original direction) and (4) re-inflating the balloon. The surgeon will thus rotate the cannula tube to align the side aperture, width axis and radial line along a second plane intersecting the vertebral body. With the catheter tube rotated to point the side aperture and balloon width axis toward a second area of the cancellous bone, the surgeon will re-inflate the balloon to create an additional intraosseous cavity, or to enlarge the initial intraosseous cavity centered on the second intersecting plane cross section.

The surgeon may then repeat the deflation and re-orientation and re-inflation as necessary to achieve an intraosseous cavity or cavities suitable for kyphoplasty. FIG. 9 shows a possible result of three inflations, with a three lobed cavity, including the original lobe 18 and additional lobs 19 and 20. With the vertebral body jacked up from the inside, to approximate its original height (its height prior to collapse) to the satisfaction of the surgeon, the surgeon will withdraw the balloon catheter from the vertebral body but leave the trocar in place to maintain access to the cavities. Promptly after removal of the balloon catheter, the surgeon will inject cement into the cavity or cavities created with the balloon using a catheter inserted through the trocar. After injecting of the cement, the surgeon will remove the catheter used for injection. The cement used for filling the void or voids (polymethylmethacrylate (PMMA) bone cement, for example) typically sets quickly.

Several features may be included in the system to enhance and facilitate use of the system and accomplishment of the method. FIG. 1 also shows, at the proximal end 2p, an indicator 26, indicative of the orientation of the side aperture. As illustrated the indicator takes the form of a flat tab 27 extending radially (outwardly from the center tube) from the catheter tube and aligned circumferentially (around the circumference of the tube) with the side aperture, such that a surgeon can be sure of the direction of the side aperture by viewing the indicator, and can direct the aperture in a desired direction by twisting the catheter tube in the desired direction. Any form of indicator, including indicia/markings on the catheter tube, may be used as the indicator.

FIG. 1 also shows a pair of locking mechanisms 28 and 29, such as compression fittings, for longitudinally and rotationally locking the catheter tube to the trocar used to insert the catheter tube into a vertebral body. These may be used in the method described above. After advancing the balloon into the cancellous bone and confirming it is positioned as desired, the locking mechanism 28 can be operated by the surgeon to longitudinally and rotationally lock the balloon catheter to the trocar, and thus prevent inflation of the balloon from displacing the catheter tube, causing malposition of the balloon and an undesirable result, or potentially causing the balloon to recede back toward the trocar potentially harming the integrity of the balloon itself (i.e. breaking the balloon). The locking mechanisms, which comprise means for locking the catheter tube to the cannula, may comprise telescope tube clamps, tube friction locks, clutch locks, split collar locks, etc. Together with the cannula, the locking mechanism and the cannula, comprise means for locking the catheter tube to the vertebral body.

A suitable locking mechanism is illustrated in FIG. 11, comprising a first locking member 28 for locking the balloon catheter relative to the second locking member 29. The second locking member comprises internal threads or other means for locking this component to the trocar proximal end. The first locking member may be threaded onto the second locking member so that rotation and translation of the first member will operate to close a split collar 30 upon the catheter tube.

In performing the method, after an inflation and creation of a first cavity, the balloon may be deflated, the locking mechanism may be released to allow twisting of the catheter tube. The surgeon may then rotate the catheter tube to redirect the side aperture, and optionally translate the catheter tube within the trocar to adjust the longitudinal position of the balloon, and then operate the locking mechanism to prevent inadvertent longitudinal or rotational movement of the catheter tube. With the balloon and side aperture directed in a new direction, the surgeon can re-inflate the balloon to create a second cavity or enlarge the first cavity.

FIG. 1 also shows a three-way valve secured to the proximal end of the cannula tube. The three-way valve 31 (a ball valve or plug valve or other) is configured to establish fluid communication between the lumen 6 and the insufflator port 32 or the drain port 33. With manual operation of the valve, a surgeon can operate the valve to place it in the insufflation configuration to supply insufflation fluid from the insufflator to the catheter tube lumen and balloon, and the surgeon may operate the valve to place it in a drainage configuration in which the catheter lumen is in fluid communication with the drain port. With the three-way valve, the surgeon can easily accomplish the steps of supplying fluid to inflate the balloon and then draining fluid from the balloon to deflate the balloon repeatedly, without disconnecting the insufflator from the catheter tube.

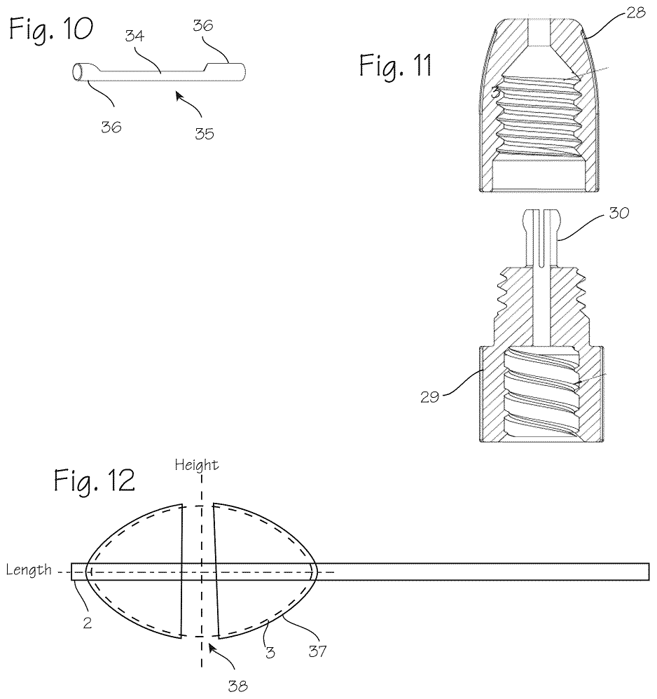

Control and support of the balloon may be enhanced with a spine or backing bar 34 behind the balloon, disposed on a luminal surface, exterior surface or inside the wall of the catheter tube, radially opposite the side aperture. This backing bar serves as a structural reinforcement of the catheter tube that prevents backward (lateral) bowing of the catheter tube upon inflation of the balloon in a direction opposite the side aperture. The backing also supports the balloon expansion in the preferred direction at higher inflation volume and pressure. When the balloon expands, the backing provides a rigid support surface that prevents expansion in its direction, ensuring the force of inflation is directed oppositely toward the intended area of vertebral height restoration. A backing bar is shown in FIGS. 2 and 10. This bar may be fixed to the catheter tube in any suitable manner. As shown, the bar is part of a backing bar assembly 35, with bands 36 fixed to the distal end of the bar and the proximal end of the bar. The bands are tightly affixed to the catheter tube at either end of the side aperture. This serves as a convenient means to affix the backing bar to the catheter tube. The bar and or band may be radiopaque to aid in visualization of the catheter tube under fluoroscopy.

The balloon may be provided with radiopaque markers to facilitate visualization of the balloon with fluoroscopy while it is positioned within the vertebral body. For example, the balloon can be entirely or partially covered with a radiopaque material or markers. In a particularly helpful arrangement illustrated in FIG. 12, radiopaque material, in the form of a coatings or layers of polymer 37, loaded with radiopaque material, is disposed on the distal end and the proximal end of the balloon, while a center portion of the balloon is not. The center strip 38 about the circumference of the longitudinal centerline is uncovered, and radiolucent. With this arrangement, the orientation of the balloon within the vertebral body can be determined under fluoroscopic visualization. The same effect may be achieved with radiopaque inner layers or coatings in the form of a coatings or layers of polymer loaded with radiopaque material on the inside of the other radiolucent outer layer, with the radiopaque layer or coating confined to a distal region of the inner surface and proximal region of the inner surface a radiolucent balloon, leaving a central region of the balloon, between the distal and proximal radiopaque portions, radiolucent.

The radiopaque layer may be applied to the balloon as a thin radiopaque tube, trimmed to create the gap, or additional gaps, then slid over a balloon blank (a parison) prior to blow-molding the blank into the balloon form in a balloon forming mold, which results in laminating the radiopaque portions of the tube to the balloon material. Alternately, two thin radiopaque tubular tubes may be slid over the balloon blank, one on each end, with a space between them in the center, prior to blow-molding the blank into the balloon form in a balloon forming mold, which results in laminating the radiopaque both portions of the tube to the balloon material, leaving the radiolucent center strip shown in FIG. 12.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A method of kyphoplasty on a vertebral body of a patient, said method comprising:
inserting a balloon into the vertebral body through a pedicle, to position the balloon in a lateral portion of cancellous bone within the vertebral body;
inflating the balloon in a first direction within the vertebral body to create a first void within the cancellous bone;
deflating the balloon;
without removing the balloon from the vertebral body, orienting the balloon in a second direction within the vertebral body;
re-inflating the balloon while oriented in the second direction to enlarge the first void;
withdrawing the balloon from the vertebral body;
injecting cement into the void.

2. The method of claim 1, comprising the steps of;
inserting the balloon into the vertebral body through a catheter tube with a side-facing aperture in the distal end of the catheter tube, wherein, in an uninflated configuration, the balloon is disposed in the catheter tube proximate the side facing aperture, and the balloon, in an inflated configuration, extends radially away from the side aperture of the catheter tube.

3. The method of claim 1, comprising the steps of:
placing the balloon into the vertebral body through a cannula inserted into the vertebral body;
locking the catheter tube to the cannula prior to inflating the balloon;
unlocking the catheter tube from the cannula prior to orienting the balloon in a second direction;
locking the catheter tube to the cannula prior to re-inflating the balloon.

4. A method of kyphoplasty comprising the steps of:
providing a kyphoplasty balloon catheter comprising a catheter tube and a balloon disposed on the distal end of the catheter tube;
wherein the catheter tube is characterized by a proximal end and a distal end, a lumen extending therethrough, and a long central axis corresponding to the center of the lumen;
the balloon, disposed on the distal end of the catheter tube, is characterized by a long axis, a width axis measured from the catheter tube and perpendicular to the long central axis of the catheter tube, and a height axis displaced from the long central axis of the catheter tube and perpendicular to the long central axis of the catheter tube and the long axis of the balloon, wherein the long axis of the balloon, when inflated, is displaced from the long axis of the catheter tube, said balloon in fluid communication with the lumen of the catheter tube;
inserting the catheter tube distal end within the vertebral body, to position the balloon inside the cortical shell of the vertebral body and amidst the cancellous bone of the vertebral body such that the long axis of the catheter tube is proximate a lateral area of the vertebral body, and the balloon is expandable toward a medial area of the vertebral body, with the width axis disposed on a first radial line relative to the catheter tube from the catheter tube central axis toward the medial area of the vertebral body, said radial line lying in a first plane transecting the vertebral body;
inflating the balloon within the vertebral body, inside the trabecular/cancellous bone, such that the balloon expands away from the long axis of the catheter tube and into an area of the cancellous bone medial to the catheter tube, to create an intraosseous cavity medial to the distal end of the catheter tube and centered on the first transverse cross section of the vertebral body;
deflating the balloon;
thereafter, without withdrawing the balloon or catheter tube;
rotating the catheter tube to align the width axis and radial line along a second plane transecting the vertebral body;
inflating the balloon to enlarge the intraosseous cavity centered on the second transverse cross section, or create a second intraosseous cavity centered on the second transverse cross section.

5. A kyphoplasty balloon catheter comprising:
a catheter tube, said catheter tube having a proximal end and a distal end, a lumen extending therethrough, and a long central axis corresponding to the center of the lumen, said catheter tube having a side facing aperture in a side aperture segment near the distal end of the catheter tube, with a partial cylinder portion opposite the side aperture;
a balloon disposed on the distal end of the catheter tube, proximate the side-facing aperture, said balloon characterized by a long axis corresponding to the long central axis of the catheter tube, a width axis measured from the catheter tube and perpendicular to the long central axis of the catheter tube, and a height axis displaced from the long central axis of the catheter tube and perpendicular to the long central axis of the catheter tube and the long axis of the balloon, said balloon having a small cross-section uninflated configuration in which it fits within the side aperture segment of the catheter tube;
said balloon having a large cross-section inflated configuration in which it extends from the side aperture segment of the catheter tube, extending radially away from the side aperture in a direction opposite the partial cylinder portion;
wherein the long axis of the balloon, when inflated, is displaced from the long axis of the catheter tube; said balloon in fluid communication with the lumen of the catheter tube.

6. The kyphoplasty balloon catheter of claim 5 wherein:
the balloon catheter is configured to limit expansion of the balloon to a minor arc about the catheter tube.

7. The kyphoplasty balloon catheter of claim 5 further comprising:
a stiffening spine secured to the partial cylinder portion of the catheter tube.

8. The kyphoplasty balloon catheter of claim 5 wherein:
the balloon comprises:
an inner layer which is radiolucent; and
a distal radiopaque polymer layer covering a distal portion of the inner layer of the balloon, said distal radiopaque polymer layer disposed around the entire circumference of the distal portion of the inner layer of the balloon;
a proximal radiopaque polymer layer covering a proximal of portion of the inner layer of the balloon, said proximal radiopaque polymer layer disposed around the entire circumference of the proximal portion of the inner layer of the balloon; and
a middle region of radiolucent balloon located between the distal radiopaque layer and the proximal radiopaque layer, said middle region disposed around the entire circumference of the middle region of the balloon.

9. The kyphoplasty balloon catheter of claim 5 further comprising:
means for locking the catheter tube to the vertebral body.
